(19) United States
(12) Patent Application Publication
(10) Pub. No.: US 2009/0298793 A1
(43) Pub. Date: Dec. 3, 2009

(54) Acylgycerophospholipids for treating symptoms concomitant with cancer

(75) Inventor: Ulrich Massing, Merzhausen (DE)

Correspondence Address: Ballard Spahr LLP, SUITE 1000, 999 PEACHTREE STREET, ATLANTA, GA, 30309-3915, US

(73) Assignee: KTB TUMORFORSCHUNGSGESELLSCHAFT MBH, FREIBURG (DE)

(21) Appl. No.: 11/989,125

(22) PCT Filed: Jul. 24, 2006
(86) PCT No.: PCT/EP2006/007275

§ 371 Date: May 12, 2009

(30) Foreign Application Priority Data
Jul. 22, 2005 (EP) 05106747.8

(51) Int. Cl. A61K 31/685

(52) U.S. Cl. 514/78

(57) ABSTRACT

The invention relates to the use of acylglycerophospholipids, in particular of hydrogenated acylglycerophospholipids and of phospholipids with a high omega-3 fatty acid content, for the production of a medicament for treating symptoms concomitant with cancer, in particular for treating tumour cachexia, cancer-related problems and pain, and for the prophylaxis of tumour growth and metastasis.

The invention relates to the use of acylglycerophospholipids, especially hydrogenated acylglycerophospholipids, and phospholipids having a high ω-3 fatty acid content for the preparation of a medicament for the therapy of symptoms concomitant with cancer, especially for the treatment of tumor cachexia, cancer-caused disorders and pain, and for the prophylaxis of tumor growth and metastatic spread.

### BACKGROUND OF THE INVENTION

Phospholipids (briefly “PL” in the following) are a main component of animal cell membranes. They usually consist of a hydrophilic head linked to hydrophobic non-polar residues through a negatively charged phosphate group. The most frequent PLs in biological membranes are glycerophospholipids.

Glycerophospholipids (“GPL” in the following) are constructed as shown in formula (I):

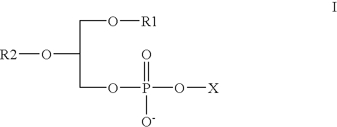

They consist of a hydrophilic head that is linked through a negatively charged phosphate group in sn-3 position with a glycerol skeleton and through this with one or two hydrophobic non-polar residues R¹ and R² (IUPAC Compendium of Chemical Technology, 2nd ed. (1997)). The latter are usually O-acyl residues consisting of fatty acids having a length of from 14 to 24 carbon atoms, but may also be O-alkyl or O-1-alkenyl residues. GPLs having one or two O-acyl residues are also referred to as 1-acyl, 2-acyl or 1,2-diacylglycerophospholipids or generally summarized as acylglycerophospholipids (briefly “AGPL” in the following). Typical acylglycerophospholipids include phosphatidylglycerol, phosphatidylserine, phosphatidylethanolamine (“PE” in the following), phosphatidylinositol (“PI” in the following), phosphatidic acid (“PA” in the following) and phosphatidylcholine (“PC” in the following). Acylglycerophospholipids are contained, in particular, in lecithin.

Due to their emulsifying effect, glycerophospholipids, especially PC or PC-containing mixtures, such as lecithin, are employed as components of food supplements and foods, in cosmetics, in parenteral nutrition and as auxiliary agents for the formulation of medicaments. Mostly, non-hydrogenated phospholipids from soybean or egg are employed, and only for the formulation of medicaments for intravenous administration, hydrogenated phospholipids are preferred.

As an alternative for native phospholipids in tumor therapy, mainly their alkyl derivatives, such as alkylphosphocholines and alkylphospholipids, or derivatives with other non-native substituents are proposed (U.S. Pat. No. 4,562,005; U.S. Pat. No. 4,775,758; EP 0 171 968; U.S. Pat. No. 5,489,580; U.S. Pat. No. 6,172,050; WO 01/72289; DE 4408011; Zeisig, R. et al., Anticancer Drug Des. 16(1): 19-26 (2001); Arndt, D. et al., Breast Cancer Res. Treat. 43(3): 237-46 (1997); DE-A-3304870; U.S. Pat. No. 4,492,659; DE-A-3204735; U.S. Pat. No. 4,565,659; Modolell, M. et al., Cancer Res. 39(11): 4681-4686 (1979)). However, the pronounced side effects (especially gastrointestinal side effects as well as negative effects on blood pressure and the blood composition) prevented the administration of amounts sufficient for tumor therapy, for example, of the promising alkylphosphocholine hexadecylphosphocholine (miltefosine) (Verveij, J. et al., J. Cancer Res. Clin. Oncol. 118: 606 (1992)), so that clinical tests with the substance (oral administration) ultimately remained without success (Verveij, J et al., Eur. J. Cancer 29A: 208 (1993); Planting, A. S. et al., Eur. J. Cancer 29A: 518 (1993)). Therefore, until today, miltefosine has been used in oncological indications only as a skin ointment for the treatment of skin metastases of mamma carcinoma (Miltex®) (Dummer, R. et al., J. Am. Acad. Dermatol. 29: 963 (1993)).

To date, administration of miltefosine by injection has not been contemplated for application in humans due to spontaneous hemolysis. The biophysical properties of miltefosine correspond to those of lysophospholipids, which exhibit membranolytical properties at high local concentrations.

A lysophospholipid is a one-chain phospholipid formed by the phospholipase A catalyzed cleavage or chemical hydrolysis of a fatty acid residue and having surface-active properties that is able to lyse red blood cells (hence the name). Lysophospholipids (1-acyl or 2-acylglycerophospholipids) can be formed from the different two-chained membrane-forming phospholipids. Examples of lysophospholipids include lysophosphatidylcholine, lysophosphatidylethanolamine (lysokephaline), lysophosphatidylglycerol, -serine and lysophosphatidic acid, whose starting phospholipids are all 1,2-diacylglycerophospholipids. Although lysophospholipids are known to be carcinostatic compounds (U.S. Pat. No. 4,372,949), they also have hemolytic properties due to their surface-active properties when the pure lysophospholipids are administered in relevant doses, just like the above discussed phospholipids that are not acylglycerophospholipids (U.S. Pat. No. 4,562,005; U.S. Pat. No. 4,775,758; EP 0 171 968; U.S. Pat. No. 5,489,580; U.S. Pat. No. 6,172,050; WO 01/72289; DE 4408011; Zeisig, R. et al., Anticancer Drug Des. 16(1): 19-26 (2001); Arndt, D. et al., Breast Cancer Res. Treat. 43(3): 237-46 (1997); DE-A-3304870; U.S. Pat. No. 4,492,659; DE-A-3204735; U.S. Pat. No. 4,565,659; Modolell, M. et al., Cancer Res. 39(11): 4681-4686 (1979)).

In addition, neither phospholipids nor lysophospholipids have so far been employed for the therapy of tumor-induced cachexia or further symptoms concomitant with a cancer disease, such as fatigue. Also, the use thereof for preventing or reducing metastatic spread or tumor growth has not been known to date.

Glycerophospholipids that are usually used for the preparation of liposomes, such as phosphatidylcholine, are even denied to have an antineoplastic effect of their own (DE-A-19959689).

Phospholipids are employed as carriers and formulation aids (emulsifiers/vesicle forming agents) for medicaments, for example, in liposomes. However, an antineoplastic effect of the phospholipids themselves has not been described so far. Thus, the use of acylglycerophospholipids in tumor or cancer therapy has been limited to their use as carriers for the formulation of active ingredients, for example, in drug-loaded liposomes (WO 99/49716).

Although EP-A-1 329 219 describes the use of dimyristoyllecithin in an agent against tumors whose effect is based on apoptosis and thus the killing of existing tumor cells. However, such an apoptotic effect is to be distinguished from antineoplastic effects, because the latter just do not refer to the killing of existing tumor cells, but to the prophylaxis against the new formation of tumor cells.

Further, phospholipids are used for the preparation of formulations for parenteral nutrition. Such formulations are emulsions and typically consist of 10, 20 or 30% of triglycerides (e.g., soybean oil, MCT (medium chain triglycerides), olive oil, fish oils or mixtures thereof (e.g. soybean/olive 4:1)). The emulsions further contain a small proportion of phospholipids (lecithin) from hen's egg as emulsifiers, where as low as possible a ratio of phospholipid to triglyceride is sought (e.g., Intralipid, Baxter, 10% (20%; 30%) emulsion: 10 g (20 g; 30 g) of soybean oil and 0.6 g (1.2 g; 1.2 g) of phospholipid/100 ml; Lipundin 10% N, Braun: 8 g of lecithin per 100 g of soybean oil; Lipofundin MCT 100/0, Braun: 8 g of lecithin per 100 g of soybean oil/MCT (1:1)). Emulsions having higher triglyceride content evidently require less phospholipid for emulsification, which is considered advantageous. At a lower phospholipid to triglyceride ratio, a better metabolic compatibility is observed with a significantly lower accumulation of phospholipids and cholesterol in the plasma (Hartig, W. et al. (Eds.), Ernäh-rungs-und Infusionstherapie, 8th Ed., Thieme (2004)). The egg lecithins employed are highly enriched lecithins having a proportion of glycerophosphatidylcholine of typically 75 or 800% (e.g., Lipoid E 75/E 80).

Cancers are usually accompanied by a number of concomitant symptoms, among which cachexia, pain and fatigue are the best known. Metastatic spread, the growth of recurrent tumors and the continued growth of an existing tumor (tumor progression) are also concomitant symptoms of cancer in the broadest sense. The relief and prophylaxis of such concomitant symptoms is the object of palliative medicine. By administering medicaments (“palliative drugs”) or other therapeutical or therapy-accompanying measures, these concomitant symptoms are to be suppressed, reduced or prevented from the start by prophylactic measures.

“Tumor cachexia” or “tumor-induced cachexia” refers to a syndrome that is frequent in tumor patients and involves a high degree of weight loss and changes of the body composition. There is reduction of the adipose tissue and of the skeleton muscles and deterioration of immunological defense mechanisms (Tisdale, M. J., Nutrition 17(5): 438-442 (2001); Tisdale, M. J., Curr. Opin. Clin. Nutr. Metab. Care 5(4): 401-405 (2002)).

De Wys et al. report that a weight loss precedes the tumor diagnosis in 31-87% of all cases, depending on the type of tumor (DeWys, W. D. et al., Am. J. Med., 491 (1980)). A severe weight loss of >10% of the healthy starting weight could be established in 150/0 of all patients when the diagnosis was made. Tumor therapies are accompanied by further losses of appetite and weight (Costa, G. and Donaldson, S. S., N. Engl. J. Med., 1471 (1979)).

Tumor-induced cachexia is a severe problem in the treatment of many cancer patients. Longitudinal studies showed that tumor patients having experienced a weight loss have a worse prognosis than those having a stable weight. Despite of stronger undesirable effects of the therapy, the tumor response is lower in such patients, and also found are a reduced physical performance, a worse evaluation of the subjective quality of life and a reduced survival (DeWys, W. D. et al., Am. J. Med., 491 (1980); Andreyev, H. J. H., Eur. J. Cancer, 503 (1998); van Eys, J., Cancer Res., 747 (1982)). In addition to sepsis, cachexia is a frequent direct cause of death in tumor patients; numerical data vary from 5 to 25% (Klastersky, J., Eur. J. Cancer, 149 (1972)).

Pfitzenmaier et al. reported that 60-70% of all patients having a progressed prostate carcinoma suffer from cachexia (Pfitzenmaier, J. et al., Cancer 97: 1211 (2003)).

Tumor-induced cachexia can be considered an inflammatory process, because increased levels of inflammatory markers are to be observed simultaneously in many of the patients in addition to malnutrition and weight loss (Moldawer, F. F. and Copeland, E. M., Cancer, 1828 (1997)). The observed release of cytokines, catabolic hormones and other regulatory peptides seems to be the primary reaction of the tumor patient's host tissues (Tisdale, M. J., Science, 2293 (2000)). In addition, other substances released by tumor cells, such as the “tumor lipid mobilizing factor” (LMF) and the “proteolysis-inducing factor” (PIF), can contribute catabolic signals and stimulate cytokine production and the acute phase reaction (Tisdale, M. J., Science, 2293 (2000)).

This systemic inflammation reaction is currently considered as the cause of the observed loss of appetite (Inui, A., Cancer Res., 4493 (1999)) and body weight (Fearon, K. C. H., World J. Surgery, 584 (1999)), which can simultaneously promote tumor progression (Coussens, L. M. and Werb, Z., Nature, 860 (2002)). Cytokine-induced metabolic changes seem to prevent the regeneration of lost body cell mass in cachectic patients, even with artificial nutrition (Espat, N. J., J. Surg. Oncol. 77, (1995)) and are associated with a reduced life expectancy (O'Gormann, P. et al., Nutrition and Cancer 36 (2000)). The resulting syndrome of reduced appetite, weight loss and inflammatory condition is referred to as cachexia, tumor cachexia or anorexia-cachexia syndrome (cancer anorexia-cachexia syndrome, CACS) (Nelson, K. A., Semin. Oncol., 64 (2000)).

In Germany, about 420,000 persons per year fall ill with cancer (numbers by the Deutsche Krebsgesellschaft (DKG) for the year 2002). According to estimations by the DKG, about 70-80% of all patients suffer from fatigue at least temporally (i.e., with only one fatigue period per patient: 315,000 patients per year). The causes for this leaden tiredness and exhaustion, which may last for from about 10 days to several months, are not clear. The cancer treatment (surgery, irradiation, chemotherapy) is essentially discussed, but further causes may include the influence of the tumor on the body as well as psychic causes.

A specific fatigue therapy has not been known to date, and it is treated by a bundle of individual strategies, such as psychic support, training (if possible) or a well-balanced diet, for example. A supplementarily balanced diet without side effects, which could alleviate or eliminate the symptoms of fatigue, would certainly be highly welcome for all patients.

A wide variety of studies proves the positive effect of ω-3 fatty acids, including EPA and DHA, in the fields of atherogenesis (lipid reduction and lowering of blood pressure), reduction of heart arrhythmia, inhibition of inflammation, PMS (premenstrual syndrome) and ADHS (attention deficit hyperactivity syndrome). Especially due to their anti-inflammatory effect, ω-3 fatty acids are also referred to as “anti-inflammatory fatty acids”.

ω-3 fatty acids also deserve particular attention in connection with AGPLs. They are usually obtained from fish oils or delivered to patients in the form of fish oils (triglycerides). However, when ω-3 fatty acids are ingested in the form of triglycerides (fish oils), there is only a slow accumulation of DHA (docosahexaenoic acid) and EPA (eicosapentaenoic acid) in the target cells/target tissues and thus a late onset of the sought effect (e.g., displacement of arachidonic acid by DHA/EPA) and of the resulting therapeutical effect. The reason for this is the processing of the triglycerides after oral administration. Namely, they are first decomposed in the gastrointestinal tract into free fatty acids and monoglycerides. The latter are then taken up by the intestinal epithelial cells and then predominantly converted back to triglycerides, which are then employed for the construction of chylomicrons. The latter arrive in the lymphatic system, then in the blood, then predominantly in the liver. In the meantime, part of the triglycerides is hydrolyzed, and the fatty acids released thereby are taken up by tissue cells and, in particular, by adipocytes (deposition). In the liver, the chylomicrons are degraded and converted to VLDLs, which are released into the blood. The latter again lose free fatty acids in the blood (triglyceride hydrolysis and uptake of the fatty acids by tissue cells and adipocytes) and are converted to LDLs. The LDLs in turn are taken up by various tissues (via LDL receptors) and degraded in the cells.

Thus, fatty acids administered as triglycerides are mainly employed for energy purposes. Only a small fraction of these fatty acids is used for the biosynthesis of phospholipids. Thus, for example, when fish oils are administered, a plateau with respect to DHA and EPA incorporation into lymphocyte membranes can be observed only after about 6-8 weeks.

Therefore, an object of the present invention is to provide another dosage form which renders ω-3 fatty acids more quickly available.

Thus, the object of the invention was to develop a medicament which contains a phospholipid as an active substance and is suitable for the therapy of symptoms concomitant with cancer, such as tumor cachexia, and for the prophylaxis of metastatic spread and tumor growth (of recurrent and primary tumors).

### SUMMARY OF THE INVENTION

It has now been found that acylglycerophospholipids, especially hydrogenated acylglycerophospholipids (preferably AGPL with solely saturated acyl residues) and AGPLs having a high content of ω-3 or ω-9 acyl residues, above all hydrogenated PC, have an antineoplastic and antimetastatic effect and reduce tumor-induced cachexia. This is true, more particularly, of AGPLs having long-chain acyl residues (chain lengths of more than 16 carbon atoms).

In addition, it has been found that ω-3 fatty acids administered in the form of acylglycerophospholipids are brought to their site of activity in the body substantially more quickly and with substantially less losses as compared to administration in the form of fish oils. Thus, the efficiency is increased. The therapeutical effect can start much more quickly. Applications in which supplementation with ω-3 fatty acids has been very difficult to achieve to date due to the large amounts to be supplied (>2-3 g of fish oil) become accessible.

A similar effect as that of AGPLs, which are rich in ω-3 or saturated acyl residues, can be achieved by AGPLs having a high proportion of ω-9 acyl residues.

Phospholipids rich in ω-3 fatty acid residues and hydrogenated acylglycerophospholipids preferably reduce tumor-induced cachexia and fatigue as well as tumor-induced pain, tumor growth and metastatic spread.

Thus, the invention relates to

(1) the use of one or more acylglycerophospholipids (briefly “AGPL” in the following) as active substances for the preparation of a medicament for the therapy or prophylaxis of symptoms concomitant with cancer;
(2) a preferred embodiment of (1), wherein said AGPL has a high proportion of saturated acyl residues, ω-3 and/or ω-9 fatty acid residues;
(3) a preferred embodiment of (1) and (2), wherein said AGPLs include only saturated acyl residues and are, in particular, phosphatidylcholines with saturated acyl residues; and
(4) another preferred embodiment of (1) to (3), in which said medicament is suitable for the treatment of symptoms concomitant with cancer.

In a particularly preferred embodiment of (1) to (3), said medicament or said AGPLs are suitable for the therapy of tumor cachexia, tumor-induced pain conditions, tumor-induced fatigue, for the prophylaxis or reduction of tumor growth and as antimetastatic agents, especially for the therapy of tumor cachexia.

The invention also relates to

(5) a process for the therapy or prophylaxis of symptoms concomitant with cancer in a patient, comprising the administration of one or more of the AGPLs as defined in embodiments (1) to (3); and
(6) a food supplement containing one or more of the AGPLs as defined in embodiments (1) to (3).

### BRIEF DESCRIPTION OF THE FIGURES

The invention is further illustrated by the following Figures in the detailed description of the invention.

FIG. 1: Effect of dipalmitoylphosphatidylcholine (DPPC) on tumor-induced cachexia in naked mice bearing a human kidney cell carcinoma (RXF 486). Dots: control without lipid (n=3); circles: 33 mg of DPPC/kg/day (days 0-4, 7-11, 14, 17, 18), oral application (n=3); rectangles define the days on which DPPC was administered; p: <0.001.

FIG. 2: Effect of hydrogenated PC on tumor progression in human soft tissue sarcoma on naked mice. Dots: control without lipid; circles: 840 mg of EPC-3/kg/week (administered on days 0, 7 and 14 by i.v. administration into the caudal vein; n=6).

FIG. 3: Effect of hydrogenated PC on tumor-induced cachexia and tumor weight in immunocompetent mice bearing a kidney cell carcinoma (RENCA model, orthotopic model). Group 1: 50 mg/kg/day of EPC-3 (n=5); group 2: untreated control (n=6).
A) Weight. Group 1: 84.3±4.3% of initial weight, group 2: 76.9±4.2% of initial weight.
B) Tumor weight (p: 0.227).

FIG. 4: Lysophosphatidylcholine level in the serum of tumor patients with and without tumor cachexia. Group 1: tumor patients without weight loss since first diagnosis; group 2: tumor patients with less than 100% weight loss since first diagnosis; group 3: tumor patients with more than 100% weight loss since first diagnosis (highly cachectic).

FIG. 5: Course of weight of patients upon administration of hydrogenated phospholipids; cf. Example 7.

FIG. 6: Lysolipid decrease in the medium of prostate carcinoma cell cultures (cf. Example 10). Cell lines employed: A) LNCaP; B) DU145; C) PC3.
The x axis shows the incubation time in hours, and the y axis shows the percent decrease of lysolipids in the medium.

### DETAILED DESCRIPTION OF THE INVENTION

The use according to the invention of AGPL as active substances in a medicament has shown to be a surprisingly effective method for the therapy of symptoms concomitant with cancer, especially tumor-induced cachexia, tumor-induced fatigue, tumor-induced pain conditions, and for the prophylaxis of metastatic spread and tumor growth (tumor progression). Thus, the medicament according to the invention is employed for the palliative treatment of cancer patients. Thus, the present invention shows, in particular, that the administration of AGPL can reduce tumor growth and tumor cachexia. Mainly hydrogenated AGPLs and AGPLs rich in ω-3 fatty acid residues reduce tumor-induced pain, cachexia and fatigue as well as tumor progression and metastatic spread.

In the following, some of the terms employed are first further defined:

An “acylglycerophospholipid” (“AGPL”) within the meaning of the present invention is a 1,2-diacylglycerophospholipid, 1-acylglycerophospholipid or 2-acylglycerophospholipid with saturated or unsaturated acyl residues, including mixtures of these three classes of substances and their pharmaceutically acceptable salts. Thus, phosphatidylcholine, lysophosphatidylcholine and lecithin belong to the AGPLs within the meaning of this definition. AGPL preferably has the structure of formula (I)

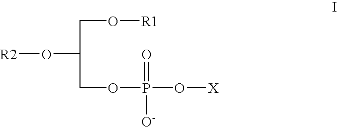

wherein

R¹ and R² are independently selected from H, alkylcarbonyl, alkenylcarbonyl, alkynylcarbonyl, arylalkylcarbonyl and cycloalkylcarbonyl residues, wherein said alkyl residues may be linear, branched or cyclic, saturated or unsaturated, and may be substituted with 1 to 3 residues R³, and one or more of the carbon atoms in the alkyl residues may be replaced by O or NR⁴;

X is selected from H (the compound then being a PA), —(CH₂)ₙ—N(R⁴)₃⁺ (this class of compounds includes PE and PC), —(CH₂)ₙ—CH(N(R⁴)₃⁺)—COO— (this class of compounds includes PS) and —(CH₂)ₙ—CH(OH)—CH₂OH (this class of compounds includes PG), wherein n is an integer of from 1 to 5;

R³ independently of the occurrence of further R³ residues is selected from H, lower alkyl (wherein said lower alkyl residues may be linear, branched or cyclic, saturated or unsaturated), F, Cl, CN and OH; and

R⁴ independently of the occurrence of further R⁴ residues is selected from H, CH₃ and CH₂CH₃;

or a pharmacologically acceptable salt thereof.

The acyl residues R¹ and R² are preferably alkylcarbonyl residues or alkenylcarbonyl residues, more preferably fatty acid residues, linear and unbranched fatty acid residues being even more preferred. The acyl residues may be saturated or unsaturated and of the same or different lengths, preferably having chain lengths of from C10 to C24, more preferably chain lengths of from C14 to C22, even more preferably from C16 to C22. Preferably, the AGPLs used according to the invention contain acyl residues selected from the group consisting of saturated acyl residues, ω-3 and ω-9 acyl residues and mixtures thereof. Unsaturated acyl residues are preferably selected from ω-3 and ω-9 fatty acid residues, especially from oleic acid (18:1), α-linolenic acid (18:3), eicosapentaenoic acid (20:5; EPA) and docosahexaenoic acid residues (22:6; DHA). On the other hand, unsaturated acyl residues preferably are not ω-6 fatty acid residues. In particular, AGPLs with saturated acyl residues more preferably contain only saturated acyl residues having a chain length of C16 or longer.

Preferred for the use according to the invention are AGPLs with naturally occurring head groups, especially phosphatidylcholines, phosphatidylethanolamines, phosphatidylglycerols, phosphatidylserines and phosphatidic acids, more preferably AGPLs selected from the group of phosphatidylcholines. In particular, phosphatidylcholines with hydrogenated or exclusively saturated acyl residues are preferred.

In a particularly preferred embodiment of (1), compounds with the structure of formula (I) or their pharmaceutically acceptable salts are used in which

(i) the acyl residues R¹ and R² independently have from 10 to 24 carbon atoms, are saturated or contain one or more double bonds, wherein the number of carbon atoms is preferably a multiple of 2, and the double bonds are not conjugated, and wherein the acyl residues are more preferably fatty acid residues;
(ii) the lower alkyl residues have from 1 to 3 carbon atoms and are preferably saturated; and
(iii) n is an integer of from 1 to 3.

Even more preferred are compounds of formula (I) in which

(i) R¹ and R² are independently H or unbranched and unsubstituted acyl residues which are either saturated (alkylcarbonyl residues), in which case they are preferably selected from lauryl (n-dodecanyl), myristyl (n-tetradecanyl), palmitoyl (n-hexadecanyl), stearyl (n-octadecanyl), arachinyl (n-eicosanyl), behenyl (n-docosanyl) and lignoceryl (n-tetracosanyl) residues, preferably selected from myristyl, palmitoyl, stearyl and arachinyl residues; or unsaturated (alkenyl- or alkynylcarbonyl residues), in which case they are preferably selected from ω-3 and ω-9 fatty acid residues, more preferably from oleyl (18:1), α-linolenyl (18:3), eicosapentaenyl (20:5) and docosahexaenyl (22:6) residues;

### (ii) R³ is H; and

(iii) X is —(CH₂)ₙ—N(CH₃)₃⁺, —(CH₂)ₙ—NH₃⁺, or —CH₂—CH(NH₃⁺)—COO—.

More preferred are AGPLs of formula (II)

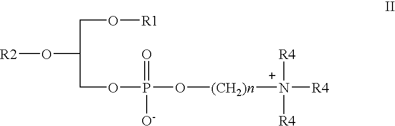

wherein
(i) R¹ and R² are independently H or unbranched and unsubstituted acyl residues which are either saturated, in which case they are preferably selected from palmitoyl, stearyl, arachinyl, behenyl and lignoceryl residues, more preferably selected from palmitoyl, stearyl and arachinyl residues; or unsaturated, in which case they are preferably selected from oleyl, α-linolenyl, eicosapentaenyl and docosahexaenyl residues;

### (ii) R⁴ is CH₃ or H; and

(iii) n is 2 or 3.

The AGPLs in the medicament according to the invention and in therapy methods in which this medicament may be employed according to the invention preferably contain a minimum amount of saturated, ω-3 or ω-9 fatty acid residues or a mixture of such fatty acid residues. This minimum amount is defined as follows:

(i) for diacyl-GPL, at least one of the two acyl residues is a saturated, ω-3 or ω-9 fatty acid residue; or
(ii) for AGPLs from natural sources, lyso-AGPLs or mixtures of one or more AGPLs, at least 50%, preferably at least 70%, more preferably at least 98% of the acyl residues contained in the AGPLs are saturated, ω-3 or ω-9 fatty acid residues. Even more preferred are AGPLs which exclusively have saturated, ω-3 and/or ω-9 acyl residues.

Among the diacyl-GPLs, those are preferred which have more than 70%, preferably more than 98%, more preferably exclusively, saturated, ω-3 and/or ω-9 acyl residues having a chain length of C16 and/or C18.

In contrast, if the AGPL is a lyso-PL, the acyl residue is preferably

(i) a saturated fatty acid residue having a length of at least C10, preferably at least C16, more preferably at least C20; or
(ii) an ω-3 or ω-9 fatty acid residue which preferably has a length of at least C16, more preferably C20, and, in particular, is an eicosapentaenoic acid residue (20:5) or docosahexaenoic acid residue (22:6).

Even more preferred is phosphatidylcholine (PC) that exclusively contains saturated fatty acid residues, ω-3 and/or ω-9 fatty acid residues.

A first preferred aspect of the present invention is the use of AGPLs containing saturated acyl residues. This includes hydrogenated AGPLs, which can be prepared by the hydrogenation of natural or synthetic AGPLs. In this use, those AGPLs are preferred which exclusively contain hydrogenated or saturated acyl residues. More preferably, hydrogenated AGPLs are used.

Further preferred in this aspect are AGPLs that are PCs with saturated acyl residues, especially dipalmitoylphosphatidylcholine (DPPC). DPPC is contained, inter alia, in lecithin, mainly in hydrogenated lecithin. For this reason, inter alia, lecithin, preferably hydrogenated lecithin, is suitable for the use according to the invention. One particularly suitable lecithin is egg or soybean lecithin, mainly hydrogenated egg or soybean lecithin.

A second preferred aspect of the present invention is the use of AGPLs that contain ω-3 and/or ω-9 fatty acid residues. More preferred are AGPLs with ω-3 fatty acid residues.

Finally, a third preferred aspect of the present invention is the use of AGPLs containing a mixture of saturated, ω-3 and ω-9 fatty acid residues.

The origin of the AGPLs (synthetic or isolated from natural sources) is irrelevant to the use according to the invention. AGPLs according to the invention also include lysoacylglycerophospholipids, which are distinguished from AGPLs having two acyl residues by lacking one acyl residue in the sn-1 or sn-2 position. Commercially available mixtures of glycerophospholipids or fractions of such mixtures may also be employed. One example thereof is the so-called lecithin, which must at least contain 20% phosphatidylcholine. Such commercially available mixtures are then hydrogenated with usual methods to prepare the hydrogenated PLs that are preferably used for performing the invention.

“Active substances” within the meaning of the present invention are compounds that can cause a physiological reaction in living organisms, especially in humans or animals. In particular, they are active substances employed in therapy. A “pharmacologically active substance” means a compound which, as an ingredient of a medicament, is the cause for its activity.

A “palliative treatment” means a therapy with a palliative objective, i.e., in cancer therapy, the alleviation or prophylaxis of tumor-caused symptoms. Such symptoms include not only tumor-caused fatigue, tumor-caused pain conditions and tumor cachexia, but also metastatic spread and tumor growth. The latter is to be inhibited or reduced by the palliative treatment, which is effected, for example, by palliative irradiation in conventional tumor therapy.

Unless otherwise stated, “tumor growth” (synonymous: tumor progression) refers to the growth of any tumor, whether it is a primary tumor, recurrent tumor or metastatic spread.

A “triglyceride” is a triester of glycerol with the same or different, saturated or unsaturated acyl residues having from 10 to 30 carbon atoms. Preferred triglycerides for use according to the invention in combination with the AGPLs are triglycerides containing (preferably exclusively) saturated fatty acid residues and those unsaturated fatty acid residues that in vivo cannot be converted to eicosanoids, especially to the highly biologically effective eicosanoids of the ‘2 series, such as PGE₂. Examples thereof are saturated/hydrogenated triglycerides, MCTs, fish oils and oils from the microalga Ulkenia (Ärztezeitung of Aug. 26, 2004), both of which contain a large amount of EPA and DHA, olive oils, rapeseed oils, evening primrose oils and linseed oils.

Omega-minus-3 fatty acids (synonymous: ω-3 fatty acids) are unsaturated fatty acids whose outermost double bond is at the last C—C bond but two as seen from the carboxyl group. ω-3 fatty acid residues in the AGPLs that are particularly preferred according to the present invention are α-linolenic acid, EPA (20:5) and (22:6) residues, EPA and DHA residues being more preferred. Omega-3 fatty acids mainly occur in fish, especially in cold water fish. Therefore, in the context of the present invention, these are the preferred natural source of ω-3 fatty acids and PLs containing ω-3 fatty acids. Omega-3 fatty acids are essential fatty acids.

Like hydrogenated fatty acids, ω-3 fatty acids cannot be converted to highly inflammatory eicosanoids in vivo either. At most, they are converted to less inflammation-promoting eicosanoids, such as PGE₃. This is why ω-3 fatty acids are considered anti-inflammatory and anti-tumoral. Thus, a shift in the fatty acid composition of a cell, especially of the cell membrane, towards increased ω-3 fatty acid proportions can act against inflammations and serve for tumor control.

In the context of the present invention, “marine phospholipids” (MPLs) are PLs obtained from animals from aquatic habitats, or synthetically prepared PLs whose composition corresponds to the composition of such natural PLs. Preferred are MPLs which are rich in ω-3 fatty acid residues, i.e., preferably contain at least 20% by weight of ω-3 fatty acid residues, based on the total amount of lipids. Fish lecithin is more preferred. Even more preferred is MPL from fish, especially from fish liver or fish roe, mainly from salmon roe.

An omega-6 fatty acid is an unsaturated fatty acid whose first double bond is found at the sixth carbon atom counted from the end of the carbon chain (the carboxyl group being considered the beginning). Prominent representatives are linolic acid, γ-linolenic acid and arachidonic acid.

An omega-9 fatty acid is an unsaturated fatty acid whose first double bond is found at the ninth carbon atom counted from the end of the carbon chain (the carboxyl group being considered the beginning). In vivo, omega-9 fatty acids can display the same effects as described above for omega-3 fatty acids. Oleic acid is an omega-9 fatty acid. Olive oil is rich in omega-9 fatty acid triglycerides, and lecithin is rich in AGPLs containing oleyl residues.

The medicament in embodiment (1) is a palliative drug because it is employed in the palliative treatment of cancer patients. Its palliative effect includes an antineoplastic effect, i.e., it is suitable for the prevention of tumor growth.

“Symptoms concomitant with cancer” within the meaning of the present application are all symptoms that accompany a cancer, especially tumor-induced cachexia, pain and fatigue. Metastatic spread and tumor growth (whether it is the growth of recurrent tumors or the continued growth of an existing tumor) are also “symptoms concomitant with cancer” in the following. In a preferred embodiment of (1), the group of symptoms concomitant with cancer consists of tumor-induced cachexia, tumor-induced fatigue, tumor-induced pain, metastatic spread and tumor growth. More preferably, the group of symptoms concomitant with cancer consists of tumor-induced cachexia, tumor-induced fatigue, tumor-induced pain and metastatic spread, more preferably of tumor-induced cachexia, tumor-induced fatigue and tumor-induced pain. Most preferably, the symptom concomitant with cancer that can be treated by an AGPL therapy is tumor-induced cachexia.

In connection with the present invention, it is important that, especially for metastasizing tumors, which often induce cachexia, an increased level of the lipolytic enzyme sPLA2, which is mainly associated with inflammatory processes, is also often to be observed (Ogawa, M. et al., Res. Comm. Chem. Pathol. Pharmacol. 74(2): 241-244 (1991)). It is described that the proinflammatory cytokines TNFα, IFNγ, IL-1 and IL-6 induce sPLA2 (Pruzanski, W. et al., Biochim. Biophys. Acta 1403(1): 47-56 (1998); Kudo, I. and Murakami, M., Prostaglandins Other Lipid Mediat. 68-69: 3-58 (2002)).

An increased level of lipolytic sPLA2 could result in an increased cellular degradation of phospholipids, which would at first lead to the release of lysophospholipids and free fatty acids in cell membranes. sPLA2 preferably cleaves fatty acids from the 2-position of phospholipids. Frequently, arachidonic acid is one of the fatty acids released in the aPLA2 hydrolysis of membrane phospholipids.

The cell membrane degradation caused by an increased sPLA2 activity would have significant consequences for the tumor patient. Since the lysophospholipids released in the cleavage of phospholipids are further degraded by lysophospholipid lipases for the major part thereof, the cells must built new phospholipids. This is effected, on the one hand, by de novo synthesis (via CTP phosphocholine cytidyltransferase, CTT) or by the reacylation of external lyso-PC (from the serum). The latter is probably the main synthetic pathway for new phospholipids, because high lyso-PC levels as occur in the serum (about 300 μM) (Raffelt, K. et al., NMR Biomed. 13(1): 8-13 (2000); Sullentrop, F. et al., NMR Biomed. 15(1): 60-68 (2000)) inhibit CTT (Boggs, K. P. et al., J. Biol. Chem. 270(13): 7757-64 (1995)).

A consequence thereof would be that the fatty acids released in the degradation of phospholipids are converted to eicosanoids. Thus, an increased sPLA2 activity would result in an increased eicosanoid production of the cells, this eicosanoid often being PGE₂ prepared from arachidonic acid in tumor cells. PGE₂ in turn has an autokrine effect on the tumor cells and stimulates their growth. In addition, an increased PGE₂ level is associated with an increased metastasizing rate (Attiga, F. A. et al., Cancer Res. 60(16): 4629-4637 (2000)). In addition, another important implication of an increased PGE₂ level is pain sensitization in tumor patients, because PGE₂ enhances the excitability of nociceptors (Brune, K. and Zeilhofer, H. U., Biospektrum 1: 36-38 (2004)).

Another consequence of the release of fatty acids, especially arachidonic acid, could be its conversion by the cytochrome P450 enzyme CYP2J2 to form the corresponding epoxides. In a more recent study (Jiang et al., Cancer Res. 65: 4707-4715 (2005)), it was shown that the expression of CYP2J2 is upregulated in human tumors. CYP232 is an epoxygenase, which converts the substrate arachidonic acid to four different isomeric epoxyeicosatrienoic acids (EETs). EETs have an apoptosis-inhibiting effect, for they protect tumor cells from the action of tumor necrosis factors. Thus, EETs prolong the life of cancer cells. Moreover, they promote angiogenesis (Pozzi, A. et al., J. Biol. Chem. 280: 27138-27146 (2005)), mitosis and the proliferation of tumor cells. The application of hydrogenated AGPLs, AGPLs rich in ω-3 or ω-9 fatty acid residues as preferred according to the invention presumably results in a reduction of the arachidonic acid content of the tumor cell membranes, so that less tumor-promoting EETs are formed.

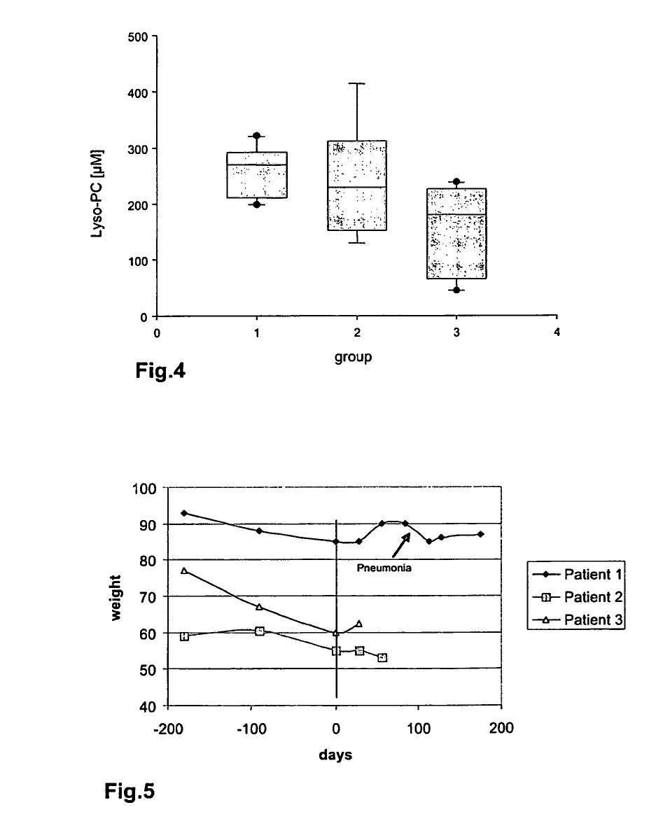

Another further consequence of the phospholipid degradation could be a depletion of lyso-PCs in the serum, if these are increasedly required for PL new synthesis. Such a depletion could be deleterious for other cells that also depend on lysophospholipids, such as the cells of the immune system. This could be a problem, in particular, with those patients who suffer from cancer anorexia-cachexia syndrome and can no longer take up sufficient food for restoring the lyso-PC level. An indication thereof is the fact that the lyso-PC level is lowered in cachectic tumor patients (group 3 in Example 4, FIG. 4).

Thus, the effect according to the invention of the AGPLs on tumor growth as well as on the symptoms tumor cachexia, metastatic spread, fatigue and pain, concomitant with cancer, are possible caused by the fact that the AGPLs reduce the accelerated cellular phospholipid degradation and the related consequences as described above, such as the release of arachidonic acid, the formation of EET and the activation of sPLA2.

Further, the effect of the AGPL according to the invention is probably based on its being cleaved into fatty acids and lysophospholipids at least partially in vivo. These two components then individually (in parallel) or commonly display an effect which could not be achieved without problems by the administration of one of the two components alone. In contrast, the administration of lyso-AGPLs could even be harmful because of its hemolytic properties and gastrointestinal side effects, while free fatty acids alone on the other hand have completely different pharmacokinetic and biophysical properties than those of AGPL and therefore could not achieve their effects in the organism.

A particularly preferred embodiment of (1) is the use according to the invention of the AGPL in combination with a triglyceride or the free fatty acids, mono- and diglycerides preparable from a triglyceride, wherein the proportion of AGPLs in the combination employed is at least 10% by weight of all lipids present. Of these, the use of the triglyceride as a further component of the medicament is preferred. The compounds mentioned preferably are or contain predominantly or exclusively hydrogenated, ω-3 or ω-9 fatty acids or fatty acid residues, and they do not contain ω-6 fatty acids or fatty acid residues, or only so in small proportions. In other words, they contain or are predominantly or exclusively fatty acid residues that cannot be converted to the biologically highly active eicosanoids in vivo. Such triglycerides or the resulting di- and monoglycerides and fatty acids include, for example, saturated/hydrogenated triglycerides, MCT, fish oils or oils from the microalga Ulkenia (Ärztezeitung of Aug. 26, 2004), both of which contain a large amount of EPA and DHA, olive oils, rapeseed oils, evening primrose oils or linseed oils.

The effect of the medicament, especially its effect on tumor-induced cachexia, can be enhanced thereby. The cause of this effect is, on the one hand, the known effect of triglycerides as a high energy food and energy supplier. On the other hand, an additional effect is obtained from the simultaneous presence of the triglycerides and AGPLs because the AGPLs, which in vivo are in part cleaved into lyso-PLs, can adopt a fatty acid residue from the triglycerides or the resulting di- and monoglycerides or free fatty acids by transesterification, whereby this fatty acid residue now is more easily incorporated into cell membranes and is not primarily transferred to energy supply (fatty acid oxidation) or deposition as a triglyceride in adipocytes. If the fatty acids incorporated in the cell membranes are predominantly hydrogenated, ω-3 or ω-9 fatty acids, the cells may be less capable of preparing PGE₂. This may in turn result in a reduction of tumor growth and metastatic spread, a reduction of pain and an improvement of the cachectic situation.

In addition, the triglycerides provide an additional reservoir of fatty acids that cannot be converted to eicosanoids. This may influence the part of cachexia that is caused by eicosanoids and thereby reduce the cachexia.

In the use according to the invention of the AGPL in combination with one or more triglycerides, it is essential that the AGPL proportion in the total lipid content of the medicament is high. Thus, this AGPL proportion should be at least 10% by weight, preferably at least 20% by weight, more preferably at least 40% by weight, of the total lipid weight. This corresponds to a ratio of AGPL:triglyceride of 1:9, preferably of 2:8, more preferably of 4:6. The reason is that the AGPLs as active substances are merely supported in their activity by the presence of the triglycerides. This also distinguishes the medicaments of the present invention from lipid preparations for artificial nutrition, in which the PL proportion must be kept as low as possible. Further, the AGPLs in combination with triglycerides can contain a broad range of fatty acid residues (hydrogenated, unsaturated) and originate from a wide variety of sources (egg, soybean, fish etc.) and thus are not limited to egg lecithin like the formulations for artificial nutrition.

Another particularly preferred embodiment is the use according to the invention of the AGPL in combination with substances having an effect on eicosanoid synthesis and thus can synergistically support the effect of AGPL. Firstly, these are PLA2 inhibitors, especially inhibitors of sPLA2 (type II sPLA) as described, for example, in Uhl et al., Phospholipase A2 Basic and Clinical Aspects in Inflammatory Diseases Vol. 24, Karger, Basel (Switzerland), pp. 123-175 (1997), and in DE-A-423-4130.

Secondly, these are substances that inhibit cyclooxygenases 1, 2 or 3, such as the non-specific compounds acetylsalicylic acid, paracetamol, diclofenac, ibuprofen, metamizol, phenazone, propyphenazone and COX-2 inhibitors, for example, meloxicam (Mobec®), celecoxib (Celebrex®), Rofecoxib (Vioxx®), Etoricoxib (Arcoxia®), Valdecoxib (Rayzon®) and Parecoxib (Dynastat®).

These substances are employed in concentrations/doses as usual for the therapeutical use of the substances or prescribed by the manufacturer.

The dose of AGPL when the medicament according to the invention is administered is preferably from 2 to 300 mg/kg/day of AGPL, more preferably from 2 to 100 mg/kg/day. The latter range applies mainly to the therapy of tumor cachexia, tumor-induced pain conditions, tumor-induced fatigue, and when the medicament is used as an antimetastatic agent. More preferably, the dose is from 2 to 40 mg/kg/day. Example 7 shows that successful results are obtained already with this dose in the therapy of tumor cachexia, tumor-induced pain conditions and tumor-induced fatigue.

The ingestion can be effected over several weeks or even months without side effects (cf. Example 7). Due to the safety to human health of the AGPLs according to the invention, even administration over one or several years is conceivable.

The medicament according to the invention is suitable for systemic administration, especially for oral (p.o.) or intravenous (i.v.) administration. Oral administration is preferred, especially if the medicament contains lyso-AGPLs. The thus administered phospholipids are cleaved into lyso-PLs and free fatty acids in the gastrointestinal tract. The free fatty acids are passed into the organism's normal fatty acid metabolism, but the lyso-PLs are not. A high proportion thereof are probably transferred directly into the blood circulation where they bind to albumin. From there, they can be taken up immediately by those cells that have a tendency to quickly taking up lyso-PLs. These include tumor cells, in particular. The incorporation in the cell membrane occurs within a rather short time.

When the medicament according to the invention is used, the known side effects of phospholipids on the blood picture in i.v. administration are prevented if 1,2-diacylglycerophospholipids are used. These substances are no micelle formers (detergents), but membrane formers, which is why they lack hemolytic properties. Therefore, 1,2-diacylglycerophospholipids are preferably used for intravenous application.

The proportion according to the invention of the acylglycerophospholipid in the total lipids in the medicament according to (1) is at least 100%, preferably at least 400/0, more preferably from 90 to 1000%. In particular, the AGPL proportion can be 1000%, the AGPL then being the only lipid in the medicament.

In a preferred embodiment of (1), the AGPL is the only phospholipid present in the medicament, more preferably the only GPL present. In the latter case, in turn, it is preferred that only acylglycerophosphocholine is present.

The AGPL may be present in the medicament as the only active substance, but it may also be combined with other active substances. In the latter case, the AGPL is preferably the only active substance from the lipids class of compounds, especially from the class of phospholipids. In a particularly preferred embodiment, however, the acylglycerophospholipid is employed as the only pharmacologically active substance contained in the medicament.

The medicament according to the invention can be formulated as usual for lipid-containing medicaments, for example, as liposomes and liposomal formulations, as a usual emulsion, as tablets, capsules, or as a powder for stirring into foods. In the preferred formulation as a tablet, the concentration of the APGLs can be up to 100%. Further preferred is the formulation as liposomes, the AGPLs being part of the liposome membrane, in which non-APGLs may also be incorporated, however. Examples of such non-AGPLs include cholesterol and negatively or positively charged amphiphiles (e.g., DOTAP).

The attending physician will adapt the dosage of the medicament individually to the respective patient. It depends, inter alia, on the kind of disease, the severity of the symptoms to be treated, the constitutional condition of the patient etc., wherein usually doses of 2-300 mg/kg of body weight/day and especially all the above mentioned preferred dose ranges are suitable.

A particular advantage of embodiment (1) resides in its importance to the preparation of medicaments for the therapy of tumor cachexia.

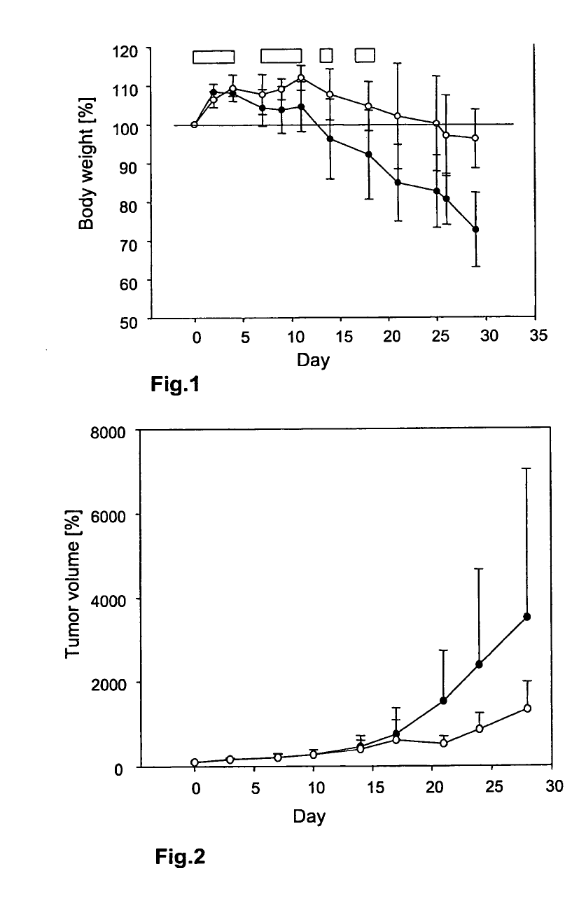

Thus, dipalmitoylphosphatidylcholine has an effect on tumor-induced cachexia. This effect is shown in Example 1 by means of the course of weight of naked mice bearing a human kidney cell carcinoma (RXF 486). This tumor has a cachexia-inducing effect. By the oral administration of a synthetic PC with hydrogenated fatty acid residues (dipalmitoylphosphatidylcholine, DPPC) as a liposomal formulation in saline, the weight loss could be virtually stopped in comparison with an untreated control group (FIG. 1). A positive effect of hydrogenated phospholipids on the course of weight in human patients with tumor-induced cachexia is also detectable (Example 7).

Another preferred aspect of embodiment (1) is the preparation of medicaments for the palliative therapy for particular tumors in humans and animals, especially for the prevention or reduction of the growth of these tumors or of metastasis formed therefrom (cf. Examples 2, 7 and 8). These are preferably tumors for which an enhanced sPLA2 expression has been observed, i.e., in particular, tumors of the gastro-intestinal tract, breast cancer, ovarian carcinoma, pancreatic carcinoma, lung cancer and above all prostate carcinoma (Ogawa, M., in: Uhl, W. et al., Phospholipase A2 Basic and Clinical Aspects in Inflammatory Diseases Vol. 24, Karger, Basel (Switzerland), pp. 200-204 (1997); Yamashita, S. et al., Clin. Chim. Acta 228(2): 91-99 (1994); Yamashita, S. et al., 3. Cancer 69(6): 1166-1170 (1994); Graff, J. R. et al., Clin. Cancer Res. 7(12): 3857-3861 (2001)). Further and particularly preferred fields of application include the palliative therapy of the symptoms including tumor growth concomitant with soft tissue sarcomas and kidney cell carcinomas.

Finally, those tumors which show a high consumption of lyso-PC in vitro and are thus suitable targets of a therapy with hydrogenated AGPLs or AGPLs containing ω-3 or ω-9 fatty acids are also preferred. These are mamma carcinomas, prostate carcinomas, pancreatic carcinomas, glioblastomas and leukemic cells, mainly prostate carcinomas (cf. Example 10).

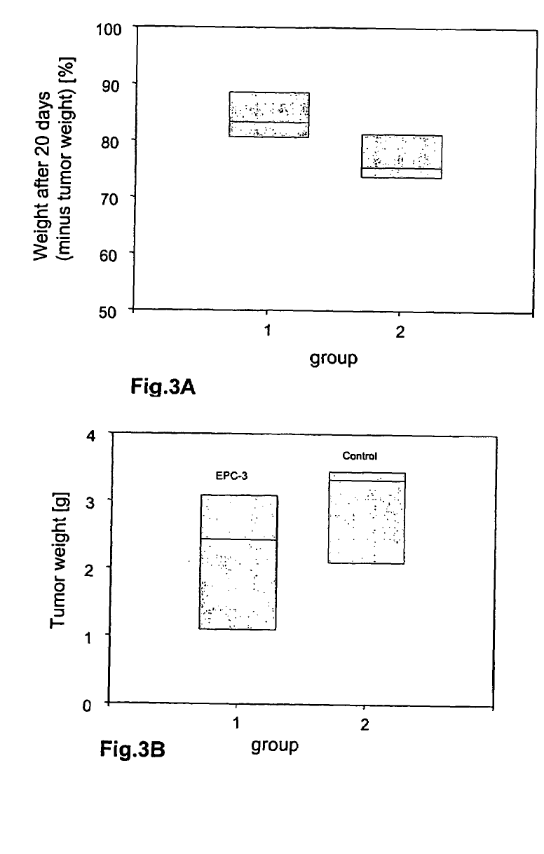

Thus, hydrogenated PC acts on the tumor progression in a human soft tissue sarcoma on a naked mouse. This tumor has no cachexia-inducing effect. By the intravenous administration of hydrogenated lecithin as a liposomal formulation with cholesterol, a deceleration of tumor growth could be achieved (Example 2; FIG. 2).

In an orthotopic tumor model, a reduction of tumor-induced cachexia associated with a reduction of tumor growth by the administration of hydrogenated egg PC is also detectable (Example 3; FIG. 3).

In one aspect of the invention, the medicament according to the invention is employed for the reduction or deceleration or even suppression of tumor growth. Preferably, the subject tumor is an existing tumor, especially a primary tumor.

It is surprising that the AGPLs cannot be employed for reducing the tumor itself. The administration of the AGPLs to cancer patients only has a palliative effect, but does not result in a reduction of the size of the tumor or killing of the tumor cells cf. Examples 2 and 7). This is where the use of the present application is distinguished from EP-A-1 329 219. EP-A-1 329 219 describes the use of dimyristoyllecithin in an anti-cancer agent that induces apoptosis. In contrast, when the medicament according to the invention is administered, no apoptosis and thus no killing of the tumor tissue takes place. Thus, liposomes of hydrogenated egg lecithin (predominantly containing C16/C18 acyl residues) do not induce cell death in tumor cell cultures (cf. Example 11 in which cultures both with and without EPC-3 addition proliferated). In a curing experiment with three final patients by the administration of S-75-3N (cf. Example 7) and hydrogenated soybean lecithin containing virtually only acyl residues with a chain length of at least C16 (analysis see Example 9), no effect on the tumor, but a weight stabilization and antidepressive effect and pain reduction (or reduction of the need for analgesics) were observed. Further, when hydrogenated egg lecithin was administered to mice with a soft tissue sarcoma, no reduction of the tumor (Example 2), but only a deceleration of tumor growth was detected.

Also, Nagami et al. (Bioorganic & Medicinal Chemistry Letters 16: 782-785 (2006)) have established that dimyristoyl-PC produces apoptosis and necrosis in tumor cells, whereas Di-C16-PC (DPPC) does not show such an effect on tumor cells.

This explains why AGPLs whose acyl residues exclusively have chain lengths of at least C16 are preferred within the scope of the present invention.

The effect of the AGPLs according to the invention, especially of hydrogenated phosphatidylcholine, on metastatic spread may also be pointed out especially (cf. Example 8). Metastatic spread, mainly in the liver, is clearly reduced or even completely suppressed by the administration of a medicament according to the invention.

Another aspect of the invention is the reduction of the need for analgesics in cancer patients to whom the medicament according to the invention is administered. This extraordinary effect was found, for example, in patient 1 in Example 7, for whom such an extraordinary result could not be anticipated in view of the advanced stage of his disease, especially not that the analgetic therapy could be temporarily discontinued altogether.

Thus, all in all, the administration of the medicament according to the invention results in a stabilization of the physical and psychical condition of the patient (no further deterioration as otherwise observed in tumor diseases, even in patients in a very late or final stage of the cancer (cf. Example 7).

If AGPLs rich in ω-3 or ω-9 fatty acid residues, such as MPLs or other AGPLs rich in ω-3 fatty acid residues, especially MPLs, are used for performing the invention, the invention has several special preferred aspects, which are set forth in the following:

In a preferred aspect of the use of AGPLs rich in ω-3 fatty acid residues, the medicament is suitable for the therapy of tumor-associated problems, especially tumor-induced cachexia, fatigue, pain conditions and metastatic spread. It is particularly suitable for the treatment of tumor-induced cachexia.

If ω-3 or ω-9 fatty acid residues in glyceride form are administered according to the invention, they are preferably in AGPL rather than triglyceride form. Preferably, AGPLs rich in ω-3 fatty acid residues are administered as components of MPLs, and AGPLs rich in ω-9 fatty acid residues are administered as components of lecithin or as dioleylphosphatidylcholine.

The AGPLs rich in ω-3 or ω-9 fatty acid residues are preferably selected from PCs, lyso-PCs, PIs and PEs, PCs being particularly preferred.

Preferred ω-3 fatty acid residues in the AGPLs rich in ω-3 fatty acid residues are long-chained ω-3 fatty acid residues, mainly with chain lengths of at least C20, more preferably DHA and EPA residues.

Preferred ω-9 fatty acid residues in the AGPLs rich in ω-9 fatty acid residues are long-chained ω-9 fatty acid residues, mainly with chain lengths of at least C18, more preferably oleic acid residues.

The AGPLs rich in ω-3 and ω-9 fatty acid residues for the use according to the invention are either synthetically prepared or are derived from natural sources. The latter case is preferred. More preferably, the AGPLs rich in ω-3 fatty acid residues are derived from marine lipid sources (“marine AGPLS”, “MPLs”), especially from animals from aquatic habitats, such as fish, mainly cold water fish. Even more preferably, they are derived from fish liver or fish roe, mainly from salmon roe. The AGPLs rich in ω-9 fatty acid residues are preferably derived from lecithin or are used in the form of lecithin within the scope of the present invention, or they are dioleylphosphatidylcholine.

The proportion of ω-3 or ω-9 fatty acid residues in the medicaments and compositions according to the invention is preferably at least 20% by weight, more preferably at least 35% by weight, even more preferably at least 45% by weight of the total amount of lipids. In a particularly preferred embodiment of the invention, the ω-3 and/or ω-9 fatty acid residues account for 50% by weight or more of the total amount of lipid in the medicament according to the invention or composition according to the invention.

Further, the weight ratio of ω-3 and/or ω-9 fatty acid residues to ω-6 fatty acid residues in the AGPLs employed is preferably at least 10:1, more preferably at least 15:1, even more preferably at least 18:1. In a specifically preferred embodiment, namely when MPLs are used, as in Examples 5 to 6, this ratio is 21:1.

AGPLs rich in ω-3 and ω-9 fatty acid residues can be employed in admixture with other substances, preferably in admixture with triglycerides, more preferably with triglycerides from the same natural source (e.g., salmon roe). The proportion of the AGPLs in the composition employed is preferably at least 5% by weight, more preferably at least 15% by weight, even more preferably at least 30% by weight of the total lipids. In the MPL employed in Examples 5 to 6, the proportion of AGPLs was 30% by weight, and it may be significantly higher in lecithin (ω-rich in 9 fatty acid residues).

Preferably, the compositions rich in ω-3 or ω-9 fatty acid residues are administered orally. The dose for patients with tumor-associated problems is preferably at least 150 mg of AGPL per day, more preferably at least 300 mg/day, even more preferably at least 450 mg/day.

A formulation and the use of the AGPLs as defined above, preferably hydrogenated lecithin and MPLs, as a food supplement is a preferred aspect of the invention. Such a food supplement is preferably employed as a supplement for the therapy of tumor-associated problems, such as cachexia, fatigue, pain conditions, and for the prevention of metastatic spread. A supplementarily balanced diet without side effects which is more capable of alleviating cachexia than the previously commercially available ω-3 products (especially fish oils) or other products should be accepted by a major part of the afflicted patients and is therefore also a preferred aspect of this invention. For even the unpleasant eructation which is often caused by fish oils does no longer take place with MPLs according to first experiences. Such a product can also be employed for prophylaxis.

The invention will be further illustrated by means of the following Examples, which do not limit the invention, however.

### EXAMPLES

### Example 1

### Effect of Hydrogenated PC on the Course of Weight of Naked Mice Bearing a Human Kidney Cell Carcinoma (RXF 486)

Tumors from the human kidney cell carcinoma RXF 486 have a string cachexia-inducing effect.

Female athymic BALB/c nude mice (nu/nu) (Charles River, Frederick, Md.) at the age of 8 to 10 weeks were employed. The animals had a weight of from 21 to 23 g and were kept under a natural day/night cycle. They were allowed water and rodent food (Altromin, Lage, Germany) ad libitum.

Fragments of 3-5 mm (in diameter) of the human kidney cell carcinoma RXF 486 were subcutaneously grafted between the animals' front and rear flanks. The therapy experiments were begun when the tumors had reached a size of 30-40 mm³.

For the therapy, synthetic PL (dipalmitoylphosphatidylcholine, DPPC; Sygena, Liestal, Switzerland) was employed. It was dispersed in 0.9% (w/v) saline in a concentration of 33 mg/ml (ultrasonic dispersion in 50 ml Falcon tube) to provide a heterogeneous liposomal dispersion. For the oral administration (p.o.), the dispersion was adjusted to 3.3 mg/ml with saline.

On experiment days 0-4, 7-11, 14, 17 and 18, the animals were treated with 33 mg each per kg of body weight of DPPC p.o. by a stomach tube (application volume: 10 ml/kg). The control animals only obtained saline. The weight of the animals was determined on days 0, 2, 4, 7, 9, 11, 14, 18, 21, 25, 26 and 29 and is shown in FIG. 1 in % of the weight on day 0.

Result: By the oral administration of the DPPC as a liposomal dispersion in saline, the weight loss could be virtually stopped as compared to an untreated control group (FIG. 1).

### Example 2

### Effect of Hydrogenated PC on Tumor Progression in a Human Soft Tissue Sarcoma on Naked Mice

Tumor from the human soft tissue sarcoma SXF 1301 have no cachexia-inducing effect.

Female athymic BALB/c nude mice (nu/nu) (Charles River, Frederick, Md.) at the age of 8 to 10 weeks were employed. The animals had a weight of from 21 to 23 g and were kept under a natural day/night cycle. They were allowed water and rodent food (Altromin, Lage, Germany) ad libitum.

Fragments of 3-5 mm (in diameter) of the human soft tissue sarcoma SXF 1301 were subcutaneously grafted between the animals' front and rear flanks. The therapy experiments were begun when the tumors had reached a size of 150-400 mm³.

Hydrogenated egg lecithin (egg phosphatidylcholine, egg PC; EPC-3 supplied by Lipoid, Ludwigshafen, Germany) was administered as a liposomal formulation with cholesterol (Merck, Darmstadt, Germany) in phosphate buffer (20 mM phosphate, 130 mM NaCl, pH 7.4) at a dosage of 840 mg/kg per week over three weeks by injection into the caudal vein of 6 animals (days: 0, 7, 14). The molar ratio of lecithin to cholesterol in the liposomal formulation was 55:45, the liposomes had a size of 40 to 60 nm. The injection volume was 10 ml/kg, the total amount of lipid administered was 2.17 mmol/kg. Control animals (n=6) received only 0.9% (w/v) saline on days 0, 7 and 14.

The tumor volumes were determined by caliper measurement (length×width²) of the subcutaneous (palpable and measurable) tumors on days 0, 3, 7, 10, 14, 17, 21, 24 and 28.

Result: By administering the hydrogenated lecithin at a dosage of 840 mg/kg/week i.v., a deceleration of tumor growth by a factor of 2 as compared to the control group could be achieved. The result is shown in FIG. 2.

### Example 3

### Effect of Hydrogenated PC on the Course of Weight and the Tumor Volume in Immunocompetent Mice Bearing a Kidney Cell Carcinoma (RENCA Model, Orthotopic Model)

The RENCA tumor had a strong cachexia-inducing effect.

Female BALB/c mice (Charles River, Frederick, Md.) at the age of 6 to 8 weeks were employed. The animals had a weight of about 20 g and were kept under a natural day/night cycle. They were allowed water and rodent food (Altromin, Lage, Germany) ad libitum.

RENCA tumor cells (murine kidney cell carcinoma) were surgically introduced in the animals by injection into the kidney (more precisely: into the subcapsular space in the left kidney). One week after the instillation of 106 RENCA tumor cells, the tumor was already macroscopically visible, and treatment of the mice was begun (day 1). At this time, the animals had a weight of from 16 to 23 g.

For the therapy, hydrogenated egg lecithin (egg phosphatidylcholine, EPC-3; supplied by Lipoid, Ludwigshafen) was employed. It was dispersed in physiological saline by ultrasound at about 50° C. to form a heterogeneous liposomal dispersion, which was adjusted to 5 mg EPC-3/ml with Ringer solution.

The animals were administered 10 ml/kg of the EPC-3 dispersion (corresponding to 50 mg/kg of EPC-3) per stomach tube p.o. on days 1-5, 8-12, 15-20. Nothing was administered to the control group.

On days 1, 3, 6, 8, 10, 13, 15, 17 and 20, the weight of the animals was measured. On day 20 after the animals had been sacrificed, the tumor weight was determined by weighing the kidney. Since it is an orthotopic tumor model and the site of the tumor was the kidney, this measurement was effected at the end of the experiment. The body weight on day 20 was corrected for the tumor weight and is represented in FIG. 3A.

Result: By the oral administration of 50 mg/kg/day of hydrogenated egg PC as a liposomal formulation in Ringer solution, the weight loss as compared to an untreated control group could be significantly reduced (p: >0.017; FIG. 3A).

Further, the EPC-3 treatment of the RENCA mice had resulted in a trend towards a lower tumor weight after 20 days (p: 0.227; FIG. 3B).

### Example 4

### Determination of the Lysophosphatidylcholine Level in Cachexia Patients

For this study, 30 patients with different solid tumors were selected. The patients either had not exhibited a weight loss since the first diagnosis (N: 11), or had exhibited a weight loss of <100% (n: 10) or a weight loss of >10% (n: 9). In the serum of these patients, the concentration of lysophosphatidylcholine (lyso-PC) was determined by means of HPTLC (high performance thin-layer chromatography). Thus, the sera (500 μl) were diluted with 500 μl of 0.9% saline and extracted three times with 2 ml each of chloroform/methanol (2:1, v/v). The combined organic phases were evaporated to dryness by means of a nitrogen flow, and the residue was taken up in 300 μl each of chloroform/methanol (2:1, v/v). By means of a Camag automated application device, 50 μl of these solutions was applied to silica gel plates (for HPTLC) (coating length 5 mm), and corresponding solutions of lysophosphatidylcholine (E. Merck, Darmstadt) in chloroform/methanol (2:1) were also applied as standards. The plates were developed in chloroform/methanol/25% ammonia/water (45/30/15/10) and subsequently dried at 180° C. The plates were dipped into a CuSO₄/phosphoric acid solution, dried (at about 60° C.) and stained at 180° C. The intensity of the lyso-PC spots was quantified by means of a Camag scanner III, and the concentration of the lyso-PCs in the patients' sera was calculated by means of the co-applied standards. FIG. 4 shows the result of the quantification.

### Example 5

### MPL-Induced Reduction of the Weight Loss of Immunocompetent Mice Bearing a Kidney Cell Carcinoma (RENCA Model, Orthotopic Model)

For this experiment, female BALB/c mice (Charles River, Frederick, Md.) at the age of 6 to 8 weeks (weight: about 20 g) were employed. Tumor cells (RENCA, murine kidney cell carcinoma) were introduced in the animals during a surgical operation by injection into the kidney (injection into the subcapsular space in the left kidney). One week after the instillation of 106 RENCA tumor cells, the treatment of the mice was begun (day 1). At this time, the mice had a weight of 19-24 g. The animals were allowed water and rodent food (Altromin, Lage, Germany) ad libitum. The RENCA tumor causes a high weight loss (cachexia-inducing).

For the therapy, marine phospholipids (MPLs) supplied by BioSea, Norway, were employed. The composition of these MPLs was as follows:

Total composition

| | |
|---|---|
| Total lipids | 99.37 g/100 g |
| Water | 0.63 g/100 g |
| Neutral lipids | 67.72 g/100 g |
| Triglycerides | 61.37 g/100 g |
| Diglycerides | 4.98 g/100 g |
| Monoglycerides | 0.99 g/100 g |
| Cholesterol | 5.04 g/100 g |
| Free fatty acids | 3.53 g/100 g |
| Polar lipids | 32.75 g/100 g |
| Phosphatidylcholine | 28.64 g/100 g |
| 2-Lysophosphatidylcholine | 0.38 g/100 g |
| Phosphatidylinositol | 0.72 g/100 g |
| Sphingosine | 1.12 g/100 g |
| Phosphatidylethanolamine | 1.89 g/100 g |

The distribution of fatty acid residues in this MPL to the neutral and polar lipids was as follows:

| | neutral | polar | |
|---|---|---|---|
| Saturated | 15.6 | 22.8 | g/100 g of lipid |
| Monounsaturated | 29.2 | 17.6 | g/100 g of lipid |
| Dienes | 2.5 | 1.3 | g/100 g of lipid |
| Polyenes | 52.6 | 58.3 | g/100 g of lipid |
| 20:5 | 18.8 | 16.5 | g/100 g of lipid |
| 22:6 | 22.8 | 33.7 | g/100 g of lipid |
| ω-3/ω-6 | 12.4 | 21.0 | g/g |

The marine phospholipids were dispersed in physiological saline by ultrasound at room temperature to form a heterogeneous dispersion, which was adjusted to 15 mg MPL/ml with Ringer solution. The animals were administered 10 ml/kg (i.e., 150 mg/kg) per stomach tube p.o. per day on days 1-5, 8-12 and 15-20. The weights of the animals were compared on the last day. The weights were corrected by the mass of the tumor.

Results: By the oral administration of MPL, 150 mg/kg/day, as a dispersion in Ringer solution, the weight loss as compared to an untreated control group could be significantly reduced (p: >0.002; group 1: 150 mg/kg/day of MPL (n=5), weight: 84.98±3.67% of the initial weight, group 2: untreated control (n=5), weight: 75.66±3.20% of the initial weight).

### Example 6

### Effectiveness of MPL on the Course of Weight of Naked Mice Bearing a Human Prostate Carcinoma (LNCaP)

For these experiments, male SCID mice (C.B-17/IcrHsd-Prkdcseid) (Harlan-Winkelmann, Borchen, Germany) at an age of from 6 to 12 weeks were used. The animals had a weight of from 25 to 35 g and were kept under a natural day/night cycle. The animals were allowed water and rodent food (Altromin, Lage, Germany) ad libitum. The animals were administered 2×10⁶ LNCaP cells in 50 μl of DMEM/matrigel (1:1) subcutaneously into the right or left flank.

The therapy experiments were begun when the tumors had reached a size of 30-40 mm³. The tumor has a cachexia-inducing effect, which requires, however, that it has reached the above mentioned size.

For the therapy of the weight loss, MPL supplied by BioSea, 150 mg/ml, was employed (thus, MPL was dispersed in saline (0.9%) by ultrasound to form a heterogeneous liposomal dispersion. For the oral (p.o.) administration, the dispersion was adjusted to 15 mg/ml with saline.

The animals were treated with 150 mg/kg MPL p.o. by a stomach tube (application volume: 10 ml/kg) over 5 weeks except for the weekends (5 days, then 2 days break). Control animals only received saline.

Result: By the oral administration of MPL as a liposomal dispersion in saline, the weight loss as compared to the control group could be reduced (group 1: 150 mg/kg/day of MPL (n=8), initial weight: 25.00±1.97 g, final weight (35 d): 20.73±1.62 (82.9±6.8%); group 2: untreated control (n=3), initial weight: 31.98±0.93 g; final weight (35 d): 25.17±1.06 (78.7±3.3%).

### Example 7

### Palliative Use of Hydrogenated Phospholipids on Patients with Advanced Tumor Diseases; Course of Weight of the Patients

On three patients with advanced metastasizing tumor disease, a curing experiment with hydrogenated phospholipids was performed. The patients received the phospholipids (S-75-3N, supplied by Lipoid) as follows: in the first 4 weeks 2×3 tablets (daily dose 780 mg/day), from the fifth week in the form of 3×3 tablets daily (1170 mg/day) (per tablet: 130 mg of phospholipid). S-75-3N is a hydrogenated phospholipid from soybean with a content of phosphatidylcholine and lysophosphatidylcholine of 81.70% in the batch employed.

The patients had different solid tumors:

Patient 1: 60 years, male, rectum carcinoma hepatically and pulmonally metastasized to the liver and lungs; the patient had received 8 different antitumoral therapies before;

Patient 2: 68 years, female, oropharyngeal carcinoma metastasized to the liver, lymph node and bones, a total of 6 different previous antitumoral therapies;

Patient 3: 74 years, male, unknown primary tumor with peritoneal carcinosis, a total of 5 previous antitumoral therapies.

Inclusion criteria: Patients were included for whom a therapy with hydrogenated phospholipids could have a positive influence on the quality of life. Pain, loss of appetite and loss of weight were defined as the outcome parameters.

All patients had suffered a significant loss of weight before the beginning of the treatment with hydrogenated phospholipid. Before the beginning of the treatment, all patients obtained an extensive anamnesis, physical examination and a complete laboratory analysis (including lipid electrophoresis). The necessary staging examinations were performed as baseline staging.

After each ingestion cycle of 4 weeks, the patients were extensively examined (follow-up). All patients received the substance as a purely supportive concept, a combination with a cytostatic therapy or other antineoplastic substances was not intended. It was taken care that an interval of about 4 weeks existed between the end of the last antitumoral therapy and the beginning of the ingestion of the phospholipids.

The patients' data are listed in Table 1. In all cases, the phospholipids were tolerated well and could be taken up continuously by the patients. Side effects were observed in none of the patients. It was found that the weight loss stopped after the ingestion of the phospholipids, and a gain of weight could even be observed with two patients (patient 1 in the second cycle of treatment, patient 2 in the first cycle). In 2 patients, the initial WHO stage (index for the quality of life) improved (patient 1: WHO 1→WHO 0; patient 3: WHO 2→1) and remained equal in patient 2 (WHO 2). Due to a severe respiratory infection, the WHO of patient 1 temporarily deteriorated to 1.

In none of the patients, the anorexia was increased by the regular intake of the phospholipids. Also, no increase of pain intensity was observed in the treatment period. In patient 1, in view of the extended liver metastasis, the very low need for analgesics in relative terms under the phospholipid therapy was particularly striking. In the 2nd and 3rd treatment cycle (ending with days 56 and 84), a regular pain medication did not even take place any more, but novalgin was administered only when needed.

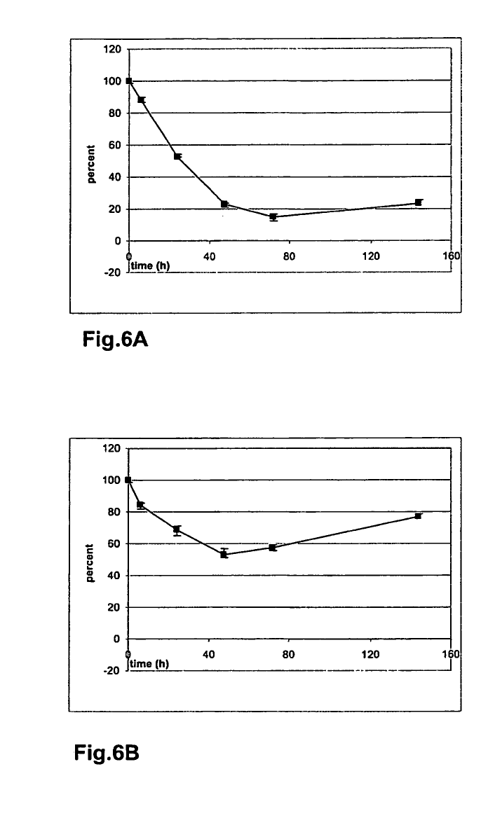

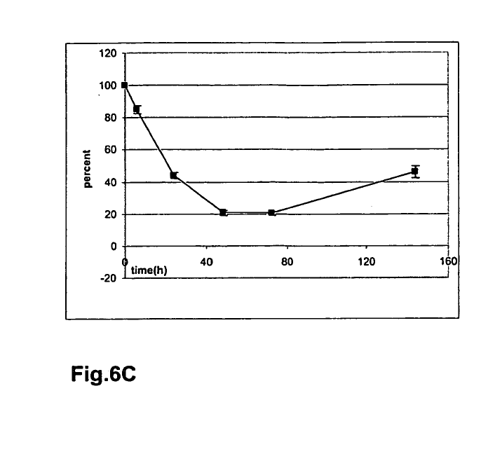

Two patients have deceased in the meantime due to their severe tumor disease (patients 2 and 3). Patient 1 still takes the phospholipids (month 5). This patient has in the meantime lost weight again due to a severe infection (see FIG. 5), but is now in an improved state again after an antibiotic therapy of the infection and gains weight again.

In patient 1, a stable course of the size of the primary tumor could be documented over 5 months clinically and by imaging.

TABLE 1
Patients' data

| Patient No. | | 1 | 2 | 3 |
|---|---|---|---|---|
| Age | | 60 | 68 | 74 |
| Sex | | male | female | male |
| Primary tumor | | Rectum | Oropharyngeal | CUP |
| | | carcinoma | carcinoma | |
| Metastases | | liver, lung | liver, lymph nodes, bones | peritoneum |
| No. of previous | | 8 | 6 | 5 |
| therapies | | | | |
| Concurrent therapies | | unconventional | unconventional, | unconventional |
| | | | Navelbine oral | |
| Start | | 13 Dec. 2005 | 19 Dec. 2005 | 05 Jan. 2006 |
| Deceased | | no | 11 Mar. 2006 | 26 Feb. 2006 |
| WHO Baseline | | 1 | 2 | 2 |
| (day 0) | | | | |
| WHO day 28 | | 1 | 2 | 1 |
| WHO day 56 | | 0 | 2 | |
| WHO day 84 | | 0 | | |
| WHO day 114 | | 1* | | |
| WHO day 128 | | 1 | | |
| WHO day 176 | | 1 | | |
| Weight −6 months | −180 | 93 | 59 | 77 |
| Weight −3 months | −90 | 88 | 60.5 | 67 |
| Weight Baseline | 0 | 85 | 55 | 60 |
| Weight day 28 | | 85 | 55 | 62.5 |
| Weight day 56 | | 90 | 53 | |
| Weight day 84 | | 88 | | |
| Weight day 114 | | 85* | | |
| Weight day 128 | | 86 | | |
| Weight day 176 | | 87 | | |

*severe infection (bronchitis) since Mar. 24, 2006; administration of Ciprobay and prednisone

### Example 8

### Antimetastatic Effectiveness of Hydrogenated Phospholipids

For this experiment, female athymic naked mice (Hsd: Athymic Nude-Foxnlnu; Harlan-Winkelmann, Borchen, Germany) at an age of 8-10 weeks were employed. The animals had a weight of about 25 g and were kept under a natural day/night cycle. The animals were allowed water and rodent food (Altromin, Lage, Germany) ad libitum.

Pieces of 1 mm3 in size of the pancreatic carcinoma MiaPaCa-2 (retrovirally transfected with the luciferase gene) were implanted orthotopically into the pancreas of the mice. After 14 days, the animals were randomized (through luciferase light reaction after injection of the luciferase substrate luciferin).

Hydrogenated egg lecithin (egg phosphatidylcholine, egg PC; EPC-3, supplied by Lipoid, Ludwigshafen, Germany) was administered as a liposomal formulation with cholesterol (Merck, Darmstadt, Germany) at a dose of 0.454 mg of EPC-3/kg of mouse weekly over 5 weeks by injection into the caudal vein of 10 animals (days: 0, 7, 14, 21, 28). The molar ratio between the lecithin and cholesterol was 55/45. The injection volume was 10 ml/kg. Control animals (n: 10) received saline only on days 0, 7, 14, 21, 28. The size of the liposomes was 36 nm on average.

The metastasis in the organs was determined by determining luciferase activity in the cell homogenizate and compared to that of the control animals.

Result: The administration of hydrogenated phospholipid at a dosage of 0.454 mg/kg/week i.v. did not cause any toxic symptoms in the animals (courses of weight identical to those of the control animals). In the animals treated with hydrogenated phospholipid, less tumor cells were seen in the livers, the main site of metastatic spread of this tumor, whereby the reduction of metastatic spread by the phospholipid administration could be shown (RLU: controls: 89.2±158.61; treated animals: 11.2±10.9; cf. Table 2). Among the treated animals, three animals had no liver metastases at all while there was only one metastasis-free animal in the control group.

TABLE 2
RLU of the livers of the control animals and of the
animals treated with hydrogenated phospholipid

| Control animals | Treated animals |
|---|---|
| Liver (RLU) | Liver (RLU) |
| 208.74 | 16.93 |
| 37.11 | 0.00 |
| 502.88 | 12.21 |
| 0.00 | 0.00 |
| 7.92 | 8.98 |
| 19.21 | 35.71 |
| 11.29 | 11.71 |
| 9.81 | 0.00 |
| 86.58 | 14.57 |
| 8.88 | 12.31 |

### Example 9

### Composition of the Hydrogenated Phospholipids Employed in the Examples (S75-3, Lyso-PC and EPC3) and Determination of their Fatty Acid Residue Composition by Gas Chromatography

The manufacturers of the hydrogenated PLS EPC-3 and S75-3-N employed here state the compositions thereof as follows:

Composition of EPC-3, batch 276015-1:

| | Specification | Result |
|---|---|---|
| Phosphatidylcholine | min. 98.0% in dry substance | 98.4% |
| Phosphatidylethanolcholine | max. 0.1% | <0.1% |
| Lysophosphatidylcholine | max. 0.5% | <0.1% |
| Sphingomyeline | max. 1.0% | <0.1% |
| Triglycerides | max. 0.5% | <0.2% |
| Cholesterol | max. 0.5% | <0.1% |
| Free fatty acids | max. 0.2% | <0.05% |
| D,1-α-Tocopherol | max. 0.1% | <0.1% |
| Phosphorus | 3.8-4.0% | 3.88% |
| Water (KF) | max. 2.0% | 1.0 |
| Ethanol | max. 0.5% | <0.05% |
| Peroxide No. | max. 3 | 0.1 |
| Iodine No. | max. 3 | 0 |
| Residual proteins | max. 50 ppm | <50 ppm |
| Heavy metals | max. 10 ppm | <10 ppm |
| Endotoxins (LAL test) | max. 6 EU/g | <1.2 EU/g |

Composition of S75-3N, batch 290077-1:

| | Specification | Result | Method |
|---|---|---|---|
| Phosphatidylcholine + | min. 70.0% | 81.7% | PC2 |
| lysophosphatidylcholine | | | |
| Phosphorus | 3.5-4.0% | 3.7% | TP |
| Water (KF) | max. 2.0% | 0.7% | USP <921> |
| Ethanol | max. 1.0% | 0.1% | ET |
| Iodine No. | max. 3.0 | 3.0 | IN |

Thus, EPC-3 consists of at least 98% PC, S75-3N consists of at least 70% PC and lyso-PC. It may be noted that no other phospholipids are contained in S75-3N.

The fatty acid residue composition was established by gas chromatography as follows:

Transesterification: In gas chromatography, the volatility of the analytes is a precondition for their determination. Therefore, the analysis of fatty acids by means of gas chromatography (GC) first requires derivatization to render them readily vaporizable. In the present case, the fatty acids were methylated before the analysis, and thus, the actual analytes are not the derivatized phospholipids, but the methyl esters of the fatty acids bound in the phospholipids. In the methyl-lation described in the following, the fatty acids bound to the phospholipids are first cleaved from the glycerol skeleton and then methylated (transesterification).

About 1 mg each of the phospholipids to be analyzed were weighed exactly and dissolved in 1 ml each of hexane (Merck, Darmstadt, Germany). The methylation was effected with methanol and added boron trifluoride, which is available as a ready-made reagent (boron trifluoride methanol solution, 14%, from Sigma, Steinheim, Germany). After the addition of 1 ml of the reagent, the mixture was heated for 1 hour at 100° C. (heating block) in a sealed glass tube. After cooling, 1 ml of distilled water was added, followed by vortexing at 1000 rpm for 5 min. Then, centrifugation was effected at 3000 rpm for 5 min. The upper hexane phase was removed with a glass Pasteur pipette and transferred to a dry and clean glass tube. The separated hexane phase was evaporated at 40° C. in the heating block under a constant nitrogen flow. The dry residue was taken up in 100 μl of hexane and again vortexed at 1000 rpm for 5 min. The solution obtained was transferred to a GC autosampler vial with an insert and sealed with a crimp cap. Then, the GC was performed (equipment: HP 5890 Series II plus Gas Chromatograph).

Analytic conditions:
capillary column with polar coating (length 50 m, ID 0.32 mm)
helium carrier gas (preliminary pressure 170 kPa)
FID detector (fuel gases hydrogen and synthetic air, make-up gas helium)
gas flow constant at 1 ml/min
split ratio 1:100
injector temperature 260° C., detector temperature 280° C.
furnace temperature program: initial 120° C. for 1 min, 30°/min to 200° C., 10°/min to 230° C., 230° C. for another 25 min
injection volume 1 μl

Evaluation: The interpretation of the chromatograph is effected by means of a standard mixture of 9 ready-made fatty acid methyl esters: C12:0, C14:0, C17:0, C16:0, C18:0, C18:1, C18:2, C18:3 and C20:0 (all from Sigma, Steinheim, Germany; the number after the colon indicated the number of double bonds contained) dissolved in hexane.

Result: The peaks for the palmitic acid methyl esters and the stearic acid methyl esters are dominant. The relative fatty acid residue composition of the phospholipids employed is shown in Table 3:

TABLE 3
Fatty acid residue composition of the PLs employed in [%]

| | S 75-3 | EPC3 | Lyso-PC |
|---|---|---|---|
| Behenic acid (docosanoic acid) | — | 3.1 | — |
| Arachic acid (eicosanoic acid) | — | 4.2 | — |
| Stearic acid | 80.0 | 61.0 | 10.0 |
| Palmitic acid | 18.6 | 30.9 | 90.0 |
| Myristic acid | <0.5 | <0.5 | — |
| Lauric acid | <0.5 | <0.5 | — |

Thus, fatty acids having a chain length of below 16 carbon atoms could be detected at only 1% of all fatty acids. In contrast, stearic acid and palmitic acid accounted for the major part of fatty acid residues in all PLs employed.

### Example 10

### Uptake of Lyso-PCs in Tumor Cells and Effect of Lyso-PCs on Such Cells

Cells: For this experiment, the following prostate carcinoma cell lines were employed: DU145, PC-3, LNCaP.

For the measurement of the lyso-PC consumption on the one hand and the measurement of the cell proliferation on the other, different culture conditions were selected:

Cell culture and incubation with lyso-PC; lyso-PC consumption: 500 μl of a cell suspension with a concentration of 2×10⁵ cells/ml were sown into the wells of a 24-well cell culture plate and incubated over night at 37° C. and 5% CO₂ (DMEM (Dulbecco's Modified Eagle Medium; Gibco Invitrogen, Eggenstein, Germany, Order No. 61965-026) plus 10% fetal calf serum (Gibco Invitrogen)). Into each well, 500 μl of a solution of 40 mg/ml albumin (BSA; bovine serum albumin, Sigma, Taufkirchen, Order No. A9418-100) and 900 μM lysophosphatidylcholine (lyso-PC, Sigma, Taufkirchen, Germany, order No. L5254, composition see Example 9) are added (final concentration in the well: 20 mg/ml albumin and 450 μM lyso-PC). The cells were incubated for another 0, 6, 24, 48, 72 or 144 h.

Cell culture and incubation with lyso-PC; cell proliferation: 100 μl of a cell suspension with a concentration of 2×10⁵ cells/ml were sown into the wells of a 96-well cell culture plate and incubated over night at 37° C. and 5% CO₂ (DMEM (Dulbecco's Modified Eagle Medium; Gibco Invitrogen, Eggenstein, Germany, Order No. 61965-026) plus 10% fetal calf serum (Gibco Invitrogen)). Subsequently, 100 μl of a solution of 40 mg/ml albumin (BSA; bovine serum albumin, Sigma, Taufkirchen, Order No. A9418-100) and 900 μM lysophosphatidylcholine (lyso-PC, Sigma, Taufkirchen, Germany, order No. L5254, composition see Example 9) are added into each well (final concentration in the well: 20 mg/ml albumin and 450 μM lyso-PC). The cells were incubated for another 0, 6, 24, 48 or 72 h.

Consumption of lyso-PC in the cell cultures: After an incubation time of 0, 6, 24, 48, 72 or 144 h, the plates were centrifuged, the supernatant removed and stored at −20° C. for the determination of the phosphatidylcholine-containing phospholipids.

The phosphocholine-containing phospholipids were determined with a commercial test, “Phospholipids B—enzymatic colorimetric method” supplied by Wako Chemicals (Neuss, Germany) according to the manufacturer's instructions. This assay is an assay for the determination of phospholipids containing phosphatidylcholine in serum or plasma. The corresponding phospholipids (phosphatidylcholine, lysophosphatidylcholine and sphingomyeline) are hydrolyzed by phospholipase D, and the free choline is subsequently converted to a betaine by a choline oxidase. The hydrogen peroxide released in this oxidation is subsequently used to couple 4-aminoantipyrine and phenol to form a colored analyte (absorption: λₘₐₓ=505 nm).

In a transparent 96-well plate, 50 μl of the cell culture supernatant and 200 μl of the reaction solution of the assay were added to each well. The reaction solution was previously prepared by adding 45 ml of buffer (50 mM Tris, 5 mg/dl calcium chloride, 0.05% phenol) to the “staining reagent” (enzyme content in 45 ml: phospholipase D 20 U, choline oxidase 90 U, peroxidase 240 U; 4-aminoanti-pyrine 0.015%). The plates were centrifuged (1200 rpm, 560×g, 1 min, to remove the bubbles) and incubated at 37° C. for 20 minutes. The absorption was determined at 490 nm, the content of PC-containing phospholipids was determined by comparison with a calibration curve (recorded with choline chloride as a reference substance).

Results: The decrease of PC-containing phospholipids in the supernatant, which are predominantly lyso-PCs due to the initial addition of 450 μM lyso-PC, is shown for the cells in FIG. 6. About 15 to 200% of the phospholipids in the supernatant originate from the cell culture media.

Determination of cell proliferation by means of BrdU assays (Roche): The BrdU assay, which is similar to the ³H-thymidine assay, was performed according to the manufacturer's instructions: 100 μl of cell culture medium was removed from each of the wells, and 10 μl of “BrdU labeling solution” was added, and incubation was performed for another four hours. During this time, the pyrimidine analogue 5-bromo-2′-deoxyuridine (BrdU) is incorporated instead of thymidine into the DNA newly formed during the cell division. The culture plates were subsequently centrifuged (room temperature (RT), 1250 rpm, 560×g, 5 min), and the cell culture medium was tapped off. The plate was subsequently dried at 60° C. for one hour, and thereafter the DNA was denatured with 100 μl each of “FixDenat solution” per well, so that the BrdU can be recognized by an antibody. The solution was again removed by tapping, 50 μl of “Anti-BrdU-POD Working Solution” was added, and the plates were incubated at RT for 90 min. The antibody-peroxidase complex contained will bind to the BrdU incorporated into the new DNA. Unbound BrdU-POD is again removed by tapping, and the wells are washed once with 200 μl and then twice with 100 μl of “Washing Solution”. An excel of liquid is removed by tapping. Subsequently, 100 μl/well of “Substrate Solution” is added, followed by incubation at RT for 20 minutes. The peroxidase reaction is stopped by the addition of 25 μl 1 NH₂SO₄, the plates are shaken for one minute (300 rpm), and the absorption is measured at 450 nm in an ELISA reader. The intensity of the color correlates directly with the DNA synthesis and is a measure of the number of proliferating cells in the culture. The more intense the color or the OD at 450 nm is, the more BrdU was incorporated, and thus the stronger the proliferation of the cells was. Results see Table 4.

TABLE 4
Results of proliferation measurement.

| Time [h] | LNCaP | PC3 | DU145 |
|---|---|---|---|
| 0 | 0.55 ± 0.05 | 1.11 ± 0.09 | 0.31 ± 0.04 |
| | 0.60 ± 0.02 | 1.16 ± 0.06 | 0.28 ± 0.02 |
| 24 | 1.30 ± 0.24 | 1.64 ± 0.13 | 0.29 ± 0.03 |
| | 1.08 ± 0.04 | 2.25 ± 0.17 | 0.22 ± 0.02 |
| 48 | 0.95 ± 0.14 | 1.64 ± 0.11 | 0.31 ± 0.03 |
| | 0.93 ± 0.05 | 1.57 ± 0.16 | 0.29 ± 0.02 |
| 72 | 1.31 ± 0.08 | 1.33 ± 0.10 | 0.39 ± 0.04 |
| | 1.09 ± 0.06 | 1.56 ± 0.11 | 0.30 ± 0.02 |

The upper value in each cell is the OD of the cells without lyso-PC addition, the lower value is the OD of the cells with lyso-PC addition.

Despite of the addition of 450 μM lyso-PC to the cell cultures and the observed uptake of lyso-PC by the cells, no increase or weakening of the growth of cell lines PC-3, DU145 and LNCaP could be seen. The measured differences are within the range of usual variations in biological systems.

### Example 11

### Effect of EPC-3 (Hydrogenated Egg Lecithin on the Proliferation of Prostate Carcinoma Cells

Cells: For this experiment, the following prostate carcinoma cell lines were employed: DU145, PC-3, LNCaP.

100 μl of a cell suspension with a concentration of 2×10⁵ cells/ml were sown into the wells of a 96-well cell culture plate and incubated over night at 37° C. and 5% CO₂ (DMEM (Dulbecco's Modified Eagle Medium; Gibco Invitrogen, Eggenstein, Germany, Order No. 61965-026) plus 10% fetal calf serum (Gibco Invitrogen)). Subsequently, 100 μl of a liposome preparation of 45 mole percent cholesterol and 55 mole percent EPC-3 (hydrogenated PC from egg, supplied by Lipoid, Ch. No. 276015-1; composition see Example 9 and xx) is added into each well (final concentration of EPC-3 in the liposomes: 55 mole percent). The liposome preparation was prepared by means of a dual asymmetric centrifuge (DAC) as described in WO 2006/069985 (Example 1 therein). The average diameter of the liposomes was 37 nm. The final concentration of EPC-3 in the wells was 24.1 μM. Into the controls, the same volume (100 μl) of 0.9% saline was added. After 48 and 72 hours of incubation, a BrdU cell proliferation assay was performed as described in Example 10.

Result: 24.1 μM hydrogenated phosphatidylcholine had no effect on proliferation in all three prostate cell lines examined. The results (OD values) are shown in Table 5. The values of the individual cell lines at the respective times are to be considered equal within the scope of usual variations of biological systems. Thus, had no influence on the cell proliferation.

TABLE 5
Representation of the influence of 24.1 μM hydrogenated phosphatidyl-
choline on the proliferation of three different prostate carcinoma cell lines
(LNCaP, PC3, DU145). The OD values from a BrdU assay are presented.

| | LNCaP | | PC3 | | DU145 | |
|---|---|---|---|---|---|---|
| Time | Control | EPC3 | Control | EPC3 | Control | EPC3 |
| 48 h | 1.00 ± 0.03 | 0.88 ± 0.10 | 0.86 ± 0.09 | 0.78 ± 0.18 | 1.43 ± 0.02 | 1.49 ± 0.03 |
| 72 h | 1.14 ± 0.19 | 1.17 ± 0.04 | 0.78 ± 0.13 | 0.80 ± 0.06 | 1.46 ± 0.10 | 1.70 ± 0.24 |

1-23. (canceled)

24. A method for the therapy or prophylaxis of symptoms concomitant with cancer in a patient, which comprises administering to said patient a composition comprising one or more acylglycerophospholipids (AGPLs) having a structure of formula (I) as an active substance 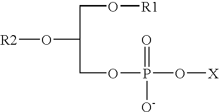
wherein
R¹ and R² are, independently of each other, selected from the group consisting of H and C₁₆₋₂₄ acyl residues, wherein the C₁₆₋₂₄ acyl residues are linear, branched, or cyclic; wherein the C₁₆₋₂₄ acyl residues are saturated or unsaturated, wherein the C₁₆₋₂₄ acyl residues are optionally substituted with 1 to 3 residues R³, and wherein one or more of the carbon atoms in the C₁₆₋₂₄ acyl residues are optionally replaced by O or NR⁴;
X is selected from H, —(CH₂)ₙ—N(R⁴)₃⁺, —(CH₂)ₙ—CH(N(R⁴)₃⁺)—COO— and —(CH₂)ₙ—CH(OH)—CH₂OH, wherein n is an integer of from 1 to 5;
R³ independently of the occurrence of further R³ residues is selected from the group consisting of H, lower alkyl, F, Cl, CN, and OH; and
R⁴ independently of the occurrence of further R⁴ residues is selected from the group consisting of H, CH₃ and CH₂CH₃;
or a pharmacologically acceptable salt thereof.

25. The method of claim 24, wherein said symptoms concomitant with cancer are one or more symptoms selected from the group consisting of tumor cachexia, tumor-induced pain conditions, tumor-induced fatigue, tumor growth, and metastatic spread.

26. The method of claim 24, wherein said AGPLs are selected from the group consisting of 1,2-diacylglycerophospholipids, 1-acylglycerophospholipids, 2-acyl-glycerophospholipids with saturated or unsaturated acyl residues and pharmaceutically acceptable salts thereof.

27. The method of claim 26, wherein said AGPLs are phosphatidylcholines.

28. The method of claim 24, wherein said AGPLs contain acyl residues selected from the group consisting of saturated acyl residues, ω-3 and ω-9 fatty acid residues and mixtures thereof, and wherein the content of acyl residues from said group in the AGPLs of the composition is at least 50% of all acyl residues contained in the AGPLs of the composition.

29. The method of claim 24, wherein said acyl residues are straight-chain and unbranched acyl residues.

30. The method of claim 24, wherein said acyl residues are saturated acyl residues.

31. The method of claim 30, wherein said AGPLs of the composition only contain hydrogenated or saturated acyl residues.

32. The method of claim 31, wherein said AGPLs are phosphatidylcholines with saturated acyl residues.

33. The method of claim 31, wherein said AGPLs are selected from the group consisting of dipalmitoylphosphatidylcholine (DPPC), hydrogenated egg lecithin and soy lecithin.

34. The method of claim 24, wherein said AGPLs are in the form of lecithin.

35. The method of claim 34, wherein said AGPLs are in the form of hydrogenated lecithin.

36. The method of claim 30, wherein the proportion of AGPLs in the total lipids of the composition is at least 10% by weight.

37. The method of claim 36, wherein the proportion of AGPLs in the total lipids of the composition is at least 40% by weight.

38. The method of claim 37, wherein the proportion of AGPLs in the total lipids of the composition is 100% by weight.

39. The method of claim 24, wherein said acyl residues are selected from the group consisting of ω-3 and ω-9 fatty acid residues.

40. The method of claim 39, wherein said acyl residues are ω-3 fatty acid residues.

41. The method of claim 39, wherein said AGPLs comprise predominantly long-chain ω-3 fatty acid residues.

42. The method of claim 41, wherein said ω-3 fatty acid residues have chain lengths of at least C20.

43. The method of claim 42, wherein said ω-3 fatty acid residues are selected from the group consisting of docosahexaenoic acid (DHA) and eicosapentaenoic acid (EPA) residues.

44. The method of claim 39, wherein said AGPLs comprise predominantly ω-9 fatty acid residues.

45. The method of claim 44, wherein said ω-9 fatty acid residues have chain lengths of at least C18.

46. The method of claim 45, wherein said ω-3 fatty acid residues are oleic acid residues.

47. The method of claim 39, wherein in said AGPLs the content of ω-3 and ω-9 fatty acid residues is at least 20% of all acyl residues contained in the AGPLs of the composition.

48. The method of claim 39, wherein the weight ratio of ω-3 fatty acid residues and ω-9 fatty acid residues to ω-6 fatty acid residues in the AGPLs of the composition is at least 10:1.

49. The method of claim 39, wherein said AGPLs are synthetic AGPLs.

50. The method of claim 39, wherein said AGPLs are marine AGPLs.

51. The method of claim 50, wherein said AGPLs are originating from marine lipid sources.

52. The method of claim 51, wherein said AGPLs are originating from animals from aquatic habitats such as fish.

53. The method of claim 52, wherein said AGPLs are originating from fish liver or fish roe.

54. The method of claim 39, wherein said AGPLs are in the form of lecithin.

55. The method of claim 39, wherein the composition, besides said AGPLs rich in ω-3 and ω-9 fatty acid residues, further comprises triglycerides.

56. The method of claim 55, wherein the proportion of the AGPLs in the composition is at least 5% by weight of the total lipids in the composition.

57. The method of claim 24, wherein said AGPLs of the composition contain a mixture of saturated acyl residues, ω-3 and ω-9 fatty acid residues.

58. The method of claim 24, wherein said AGPLs are the only pharmacologically active substance of the composition.

59. The method of claim 24, wherein the composition further contains additional components selected from the group consisting of triglycerides, free fatty acids, diglycerides and monoglycerides, and wherein the total proportion of the additional components mentioned is at most 90% by weight of all lipids present in the composition.

60. The method of claim 24, wherein the composition further contains substances having activity on the phospholipid metabolism.

61. The method of claim 60, wherein the substances having activity on the phospholipid metabolism are selected from the group consisting of CyP450 inhibitors, cyclooxygenase inhibitors and phospholipase A2 inhibitors.

62. The method of claim 59, wherein said additional component of the composition is one or more triglycerides, and wherein the ratio of AGPL to triglyceride is equal to or greater than 1:9.

63. The method of claim 24, wherein said composition is administered orally.

64. The method of claim 24, wherein said composition is administered intravenously.

65. The method of claim 24, which comprises administering the AGPLs in an AGPL dose of 2-300 mg/kg per day to said patient.

66. A food supplement comprising an AGPL or AGPL-containing compositions as defined in claim 24.

67. The food supplement of claim 66, which is suitable for accompanying a therapy for symptoms concomitant with cancer.

68. The food supplement of claim 66 which contains hydrogenated lecithin or marine phospholipids (MPLs).
